(19) United States
(12) Patent Application Publication
(10) Pub. No.: US 2004/0072282 A1
(43) Pub. Date: Apr. 15, 2004

(54) Synthesis of membrane proteins

(75) Inventors: Frank Guarnieri, Princeton, NJ (US); John Kulp III, Princeton, NJ (US); William Chiang, Princeton, NJ (US)

Correspondence Address: ALLEN BLOOM, C/O DECHERT, PRINCETON PIKE CORPORATE CENTER, P.O. BOX 5218, PRINCETON, NJ, 08543-5218, US

(73) Assignee: Sarnoff Corporation

(21) Appl. No.: 10/602,265

(22) Filed: Jun. 23, 2003

Related U.S. Application Data
(60) Provisional application No. 60/418,456, filed on Oct. 15, 2002.

(51) Int. Cl. C12P 21/06; C07K 14/47

(52) U.S. Cl. 435/68.1; 530/403

(57) ABSTRACT

Methods for chemical ligation and synthesis of peptides and polypeptides including membrane polypeptides and hydrophobic peptides and polypeptides or membrane polypeptide domains in a multiphase solvent mixture comprising at least two component solvents; compositions produced using these methods and assays performed using these compositions.

This application claims priority to U.S. Serial No. 60/418,456, filed Oct. 15, 2002.

The present invention relates to methods for the synthesis of peptides and polypeptides including membrane polypeptides and hydrophobic peptides and polypeptides or membrane polypeptide domains in a multiphase solvent mixture comprising at least two component solvents. The present invention also relates to compositions produced using the methods of the present invention and assays performed using those compositions.

Obtaining the three dimensional structure of proteins is a worldwide academic and commercial endeavor. The two major methods for solving the three-dimensional structure of a protein are x-ray crystallography and nuclear magnetic resonance. Both techniques require a large amount of material (>10 milligrams). Because of these material requirements, almost all known structures are of water-soluble proteins. Membrane proteins, which are currently the target of more than 50% of the drugs on the market, normally are expressed in Pico molar quantities and thus naturally exist in quantities substantially below the amounts needed for structure determination. Milligram quantities of rhodopsin, which is the one receptor that naturally expresses at a very high level, have been obtained using bioreactors and an insect expression system.¹ Synthetic protocols for making proteins by linking peptides in aqueous solution using native chemical ligation with n-terminal cysteines have had significant successes.² There has been one report on the synthesis of a small membrane protein using this method.³ These methods have been generalized to n-terminal glycines.⁴ This allows for selecting the naturally occurring glycines as the position where peptides are to be ligated, eliminating the need to incorporate nonnatural cysteines into the polypeptide. ¹ See, eg., Biochem J Sep. 1, 1999; 342 (Pt2): 293-300. ² See, eg., Annu Rev Biochem 2000; 69:923-60. ³ See, eg., Biochemistry Sep. 14, 1999; 38(37): 11905-13. ⁴ See, eg., PNAS Jun. 5, 2001; 98: 6554-59.

The diverse properties of proteins derive from the folded three dimensional structures of their polypeptide chains. That is, the three dimensional structure of a given protein, in conjunction with surface features, will determine its functionality. Notwithstanding, techniques have not yet been adequately developed to predict and/or fully explain the biological properties exhibited by a given protein on the basis of its three-dimensional structure alone. Rather, systemically varying the covalent structure of the protein and correlating such changes to the effects on the three dimensional structure and biological function typically determine an understanding of the structure to function relationship for a given protein. Accordingly, improved protein/polypeptide synthesis techniques are constantly being sought.

Some techniques for protein synthesis can be derived from recombinant DNA-based molecular biology. Such techniques can be used to express certain proteins in genetically engineered microorganisms. Site-directed mutagenesis⁵ has allowed for the synthesis of modified proteins in sufficient variety and quantity to facilitate systematic study.⁶ Ribosomal protein synthesis, however, appears to have certain inherent limitations.⁷ Among these limitations is the difficulty involved in producing tens to hundreds of milligrams of material by recombinant methods to make certain polypeptides. ⁵ See, eg., Smith, Angew. Chem. Int. Ed. Engl. (1994): vol. 33, p 1214. ⁶ See, eg., Eigenbrot and Kossiakoff, Current Opinion in Biotechnology (1992): vol. 3, p 333. ⁷ See, eg., Cornish, et al., Proc. Natl. Acad. Sci. USA (1994): vol. 91, p 2910.

Chemical synthesis methods have also played a roll in the exploration of the relationship between protein structure and function. Small proteins have been prepared using stepwise solid phase synthesis methods.⁸ Several full-length proteins have also been successfully synthesized using stepwise solid phase chemical synthesis methods.⁹ ⁸ See, e., Muir et al., Curr. Opin. Biotech. (1993): vol. 4, p 420. ⁹ See, e g, Miller, et al., Science (1989): vol. 246, p 1149; Wlodawer, et al., Science (1989): vol. 245, p 616; Huang, et al., Biochemistry (1991): vol. 30, p 7402; and Rajarathnam, et al., Science (1994): vol. 264, p 90.

Semi-synthetic methods have also been developed which employ the conformationally assisted relegation of peptide fragments, in certain instances, to facilitate the study of the structure/function relationship of proteins.¹⁰ Semi synthesis may also involve the use of enzymatic ligation of cloned or synthetic peptide segments.¹¹ ¹⁰ See, eg., Offord, “Chemical Approaches to Protein Engineering”, in Protein Design and the Development of New therapeutics and Vaccines, J. B. Hook, G. Poste, Eds., (Plenum Press, New York, 1990) pp. 253-282; Wallace, et al., J. Biol. Chem. (1992): vol. 267, p 3852. ¹¹ See, eg., Abrahmsen, et al., Biochemistry (1991): vol. 30, p 4151; Chang, et al., Proc. NatI. Acad. Sci. USA (1994): in press.

Chemical ligation of unprotected peptide segments has been presented as a potential tool for the synthesis of proteins.¹² Chemical ligation techniques have facilitated the preparation of purified peptides in excess of 50 amino acid residues. More particularly, using optimized stepwise solid phase methods, the preparation of high purity peptides up to 60 residues with good yield has become routine. In some instances, peptides having an excess of 80+residues have been prepared.¹³ ¹² See, eg., Schnlzer, et al., Science (1992): vol., 3256, p 221. ¹³ See, eg., Schnolzer, et al., Int. J. Pept. Prot. Res. (1992): vol. 40, p 180-193.

The conventional chemical ligation techniques rely on a chemoselective reaction to specifically and unambiguously combine peptides through the formation of an unnatural backbone structure at the site of ligation. These ligation techniques have facilitated the preparation of a variety of backbone-modified proteins, including analogues of certain protein domains.¹⁴ Chemical ligation has also been used to synthesize large quantities of high purity protein with full biological activity.¹⁵ ¹⁴ See, eg., Williams, et al., J. Am. Chem. Soc. (1994): in press. ¹⁵ See, e., Milton, et al., “Synthesis of Proteins by Chemical Ligation of Unprotected Peptide Segments: Mirror-Image Enzyme Molecules, D- & L-HIV Protease Analogs,” in Techniques in Protein Chemistry IV, Academic Press, New York, pp. 257-267 (1992).

Chemical ligation exhibits potential for use in the straightforward production of certain protein-like molecules of unusual topology. Examples of such protein-like molecules prepared using chemical ligation techniques include: (a) a four-helix bundle template-assembled synthetic protein having a molecular weight of 6647 Daltons;¹⁶ (b) a homogeneous multivalent artificial protein having a molecular weight of 19,916 Daltons;¹⁷ (c) an artificial neoprotein mimic of the cytoplasmic domains of a multichain integrin receptor having a molecular weight of 14,194 Daltons;¹⁸ and (d) a peptide dendrimer having a molecular weight of 24,205 Daltons.¹⁹ ¹⁶ Dawson, et al., J. Am. Chem. Soc. (1993): vol. 115, p 7263. ¹⁷ Rose, J. Am. Chem. Soc. (1994): vol. 3116, p 30. ¹⁸ Muir, et al., Biochemistry, (1994): vol. 33, pp 7701-7708. ¹⁹ Rao, et al., J. Am. Chem. Soc. (1994): vol. 116, p 6975.

To date, efforts to produce membrane proteins using chemical synthesis techniques have resulted in the synthesis of only one such protein—a simple 97-residue M2 protein from influenza A virus.²⁰ This lack of the chemical synthesis of any additional, more complex membrane polypeptides speaks volumes given the significant financial incentives associated with making a structure determination for such a protein. ²⁰ Kochendoerfer, et al., Total chemical synthesis of the integral membrane protein influenza A virus M2: role of its C-terminal domain in tetramer assembly, BIOCHEMISTRY Sep. 14, 1999; 38(37):11905-13.

A modular approach to synthesizing proteins was described by L. E. Canne, et al. and presented at the Annual Meeting of the Protein Society, San Diego, July 1994. Individual modules comprising protein domains of 50 to 70 residues were prepared using chemical ligation techniques. The individual modules where subsequently linked together to produce the target protein.

Notwithstanding the variety of conventional protein/polypeptide synthesis techniques, including chemically, ribosomally in a cell free protein synthesis system, or ribosomally within a cell, methods for producing a wide range of polypeptides and proteins have yet to be demonstrated.

For example, G-protein coupled receptors are integral membrane proteins that are currently the largest class of drug targets. Only one member of the family, rhodopsin has been successfully crystallized. Rhodopsin is unique to G-protein coupled receptors in that it is the only member of that family of receptors that is naturally extremely highly expressed. The other members of that family of receptors are expressed in Pico molar quantities. This lack of material in the natural systems is a major impediment to three dimensional structure determinations for these receptors. The method of the present invention provides a means for producing significant amounts of material for G-protein coupled receptors and other membrane proteins. The bases for creating a mixed solvent membrane mimetic, upon which the present invention derives, include: (1) peptides will properly fold if they exist in their native environment; (2) improperly folded peptides have a significant probability of burying their reactive sites, (3) membrane mimetic solvent systems will not only induce proper folding, but place hydrophobic regions in one phase and reactive hydrophilic regions in a different phase, (4) partitioning the reactive site into a different phase will maximize its exposure, (5) a second peptide added to this system will also partition its hydrophobic regions into one phase and the reactive hydrophilic site into a different phase and (6) this system allows for maximum exposure and collision of the two reactive sites enabling high yield chemical coupling.

Two major structural classes of membrane proteins are known. One class of membrane proteins inserts alpha-helices into the lipid bilayer. The other class of integral membrane proteins form pores in the lipid bilayer by beta-barrel strands.²¹ Single membrane spanning proteins (i.e., single-pass membrane proteins) generally have a hydrophobic region that anchors it in the lipid bilayer via an alpha-helix configuration. Multiple membrane spanning proteins (i.e., multi-pass membrane proteins) result from the polypeptide chain passing back and forth across the lipid bilayer and are typically anchored in the lipid bilayer by alpha-helix and/or beta-barrel structured membrane anchors. ²¹ See, eg., Montal, et al., Curr. Opin. Stuc. Biol. (1996) 6:499-510; Grigorieff, et al., J. Mol. Biol. (1996) 259:393-42; and Weiss, et al., J. Mol. Biol, (1992) 227:493-509.

Membrane proteins include membrane-associated receptors, transporter proteins, enzymes and immunogens. Cell membrane-associated proteins are of particular therapeutic importance. The basic superfamilies include (1) the enzyme-linked receptors, (2) the fibronectin-like receptors, (3) the seven transmembrane receptors and (4) the ion channel.

Enzyme-linked receptors comprise single-pass membrane proteins and exhibit a single polypeptide traversing the plasma lamella once via an alpha-helix anchor domain. The extracellular domain of such enzyme-linked receptors binds to a hormone/ligand. The carboxyl-terminal domain of such enzyme-linked receptors contains a catalytic site that promotes signal transduction via hormone/ligand binding and receptor aggregation.

Fibronectin-like receptors exhibit the same general structure as the enzyme-linked receptors, except that, no specific catalytic site is represented in the cytoplasmic domain. Class 1 fibronectin-like receptors contain two modified extracellular domains formed from two seven-stranded beta-sheets, which join at right angles to form a ligand-binding pocket. Class 2 fibronectin-like receptors have a slightly different structure. That is, they form repeats of five-stranded beta-sheets, which extend over the hormone like fingers. The fibronectin-like receptors of class 1 and 2 contain a conserved proline-rich cytosolic juxtamembrane region which constitutively binds soluble tyrosine kinases and which is activated by ligand/hormone-binding and receptor aggregation.

Seven-transmembrane receptors are also sometimes referred to as G-protein coupled receptors, serpentine receptors and heptahelical receptors. The seven-transmembrane receptors represent the largest and most diverse family of membrane receptors yet identified. These receptors mediate sensory and endocrine related signal transduction pathways and are multi-pass membrane proteins exhibiting alpha-helical anchor regions that transverse the membrane seven times. The transmembrane spanning regions for some of these receptors form a small ligand/hormone-binding pocket, while larger binding sites are formed through extended amino terminal regions. Seven-transmembrane receptors also contain one or more intracellular loops, which bind and activate G-proteins.

Ion channels are represented by the ligand- and voltage-gated channel membrane protein receptors. Channels may be gated by ligand binding to a receptor, by a voltage change, or by direct ligand binding if the channel also contains a binding site. Another major class of membrane proteins are the transporters, such as the serotonin transporter, which is the target of Prozac®.

Different techniques have been implemented to facilitate the study of membrane proteins and to help exploit such proteins. Notwithstanding, membrane proteins present significant obstacles including the poor solubility of their hydrophobic polypeptide chains, the difficulty in folding membrane proteins from unfolded polypeptide chains and the difficulty in overexpressing and isolating them in environments suitable for quantitative analyses. Huang, et al., J. Biol. Chem. (1981) 256:3802-3809; and Liao, et al., J. Biol. Chem. (1983) 258:9949-9955.

FIG. 1 is a representation of the dielectric properties of a cell membrane inspired by the work of White, et al., Curr. Struc. Biol. (1994) 4:79-86. The essential features are the inner 30A of constant low dielectric environment of approximately four created by the lipid chains, flanked on both sides with a variable dielectric gradient from approximately four to seventy-eight spanning 15A followed by the intracellular and extracellular aqueous phases. FIG. 2 depicts a model of penetration of a molecule through the membrane based on its position on the lipid chain. This expected penetration is the result of molecular compatibility with the dielectric environment. FIG. 3 is a representation of a G-protein coupled receptor aligned within the membrane environment. Between the horizontal parallel black lines is the 7-helical transmembrane spanning domains connected above by the extracellular loops and below by the intracellular loops. The membrane model is aligned with the molecular model of the receptor to approximately show, which part of the protein is within which part of the dielectric environment of the membrane. The four residues in black at the bottom of the helix 6 are residues that straddle the low constant dielectric variable dielectric interface.

What is needed is a method suitable for synthesizing membrane polypeptides and membrane polypeptides with site-specific chemical modifications in sufficient quantities to facilitate analyses to determine the three-dimensional structure thereof to further facilitate drug discovery efforts therewith.

### SUMMARY OF THE INVENTION

The teachings of the present invention provide multiphase solvent mixtures containing at least two component solvents that can be used as simple cell membrane mimetics. Hydrophobic peptide/protein synthesis, which is difficult to impossible with conventional synthesis techniques, can be performed easily with the multiphase solvent mixtures of the present invention. The yields obtainable using the synthesis methods of the present invention make it feasible to collect enough material to perform three dimensional structure determination with techniques such a nuclear magnetic resonance or x-ray crystallography. It is believed that the efficiency of the chemical coupling facilitated by the synthesis methods of the present invention is related to the membrane mimetic assisting in the correct folding of the polypeptide/protein, thus the resultant molecule is likely to adopt a fold related to the functionally active structure. This is believed to be important because standard detergent solubilization of membrane proteins often denatures the molecule. Also, because the synthetic methods of the present invention are based on compatibility with the dielectric environment of the cell membrane, it should be of general applicability to all membrane proteins such as ion channels, transporters, receptors and other membrane associated proteins. It is further believed that a major part of the peptides in the synthesis methods of the present invention are in a different phase from where the chemical reaction is taking place. This “phase shielding” may reduce or eliminate the need for the typical chemical procedure of protecting and deprotecting unwanted side chain reactions.

In an embodiment of the present invention, a method for the synthesis of a product polypeptide is provided, including: (a) preparing a multiphase solvent mixture, wherein the solvent mixture contains at least one organic solvent; (b) adding a first polypeptide containing a first amino acid having a first unprotected reactive group; (c) adding a ligation label containing a second amino acid having a second unprotected reactive group; and (d) collecting the product polypeptide formed by a reaction between the first polypeptide with the ligation label; wherein the first unprotected reactive group and the second unprotected reactive group react to form a covalent bond between the first unprotected reactive group and the second unprotected reactive group.

In one aspect of this embodiment, the multiphase solvent mixture is a two phase mixture.

In another aspect of this embodiment, the multiphase solvent mixture may contain at least two component solvents, wherein the dielectric strength exhibited by the at least two component solvents differ by at least 3, wherein one of the at least two component solvents exhibits a dielectric strength of 4 to 12 while another of the at least two component solvents exhibits a higher dielectric strength.

In another aspect of this embodiment, the multiphase solvent mixture may contain an emulsion. In this aspect of this embodiment, the multiphase solvent mixture may further contain an emulsifier. Emulsifiers suitable for use in the multiphase solvent mixtures of the present invention include, but are by no means limited to, phospholipids (such as lecithin), block copolymers (such as a poloxamer copolymer), and mixtures thereof.

In another aspect of this embodiment, the first polypeptide may be selected to have a sequence of membrane-incorporated polypeptide segment.

In another aspect of this embodiment, the ligation label may contain a second polypeptide that is selected to have a sequence of membrane-incorporated polypeptide segment.

In another aspect of this embodiment the first polypeptide may be selected from the group consisting of a membrane polypeptide, a transmembrane polypeptide, a receptor and useful fragments thereof.

In another aspect of this embodiment, the first polypeptide and the second polypeptide may provide portions of a G-protein coupled receptor such that the linked polypeptides form a larger portion of the G-protein coupled receptor.

In another aspect of this embodiment, the multiphase solvent mixture may contain at least two organic solvents. For example, the multiphase solvent mixture may contain DMF (Dimethyl formamide) and octanol.

In another embodiment of the present invention, a method for chemoselective chemical ligation of a product polypeptide is provided, including: (a) preparing a multiphase solvent mixture, containing at least one organic solvent; (b) adding a first polypeptide containing a first amino acid having a first unprotected reactive group; (c) adding a ligation label containing a second amino acid having a second unprotected reactive group; and, (d) collecting the product polypeptide formed by a reaction between the first polypeptide with the ligation label; wherein the first unprotected reactive group undergoes chemoselective chemical ligation with the second unprotected reactive group to form a covalent bond between the first unprotected reactive group and the second unprotected reactive group.

In one aspect of this embodiment, the chemoselective ligation is selected from the group of native chemical ligation, oxime-forming ligation, thioester-forming ligation, thioether-forming ligation, hydrazone-forming ligation, thiazolidine-forming ligation and oxazolidine-forming ligation.

In another aspect of this embodiment, the first polypeptide may be selected to have a sequence of membrane-incorporated polypeptide segment.

In another aspect of this embodiment, the multiphase mixture contains two phases.

In another aspect of this embodiment, the multiphase solvent mixture contains at least two component solvents, wherein one of the at least two component solvents exhibits a dielectric strength of 4 to 12 while another of the at least two component solvents exhibits a higher dielectric strength.

In another aspect of this embodiment, the multiphase solvent mixture may be an emulsion. In this aspect of this embodiment, the multiphase solvent mixture may further contain an emulsifier. Emulsifiers suitable for use in the multiphase solvent mixtures of the present invention include, but are by no means limited to, phospholipids (such as lecithin); block copolymers (such as a poloxamer copolymer), and mixtures thereof.

In another aspect of this embodiment, the first polypeptide may be selected to have a sequence of membrane-incorporated polypeptide segment.

In another aspect of this embodiment, the ligation label may contain a second polypeptide selected to have a sequence of membrane-incorporated polypeptide segment.

In another aspect of this embodiment, the first polypeptide may be selected from the group consisting of a membrane polypeptide, a transmembrane polypeptide, a receptor and useful fragments thereof.

In another aspect of this embodiment, the first polypeptide and the second polypeptide may provide portions of a G-protein coupled receptor such that the linked polypeptides formed from reaction therebetween give a larger portion of the G-protein coupled receptor.

In another aspect of this embodiment, the multiphase solvent mixture contains at least two organic solvents. For example, the multiphase solvent mixture may contain DMF (Dimethyl formamide) and octanol.

In another embodiment of the present invention, a method of assembling polypeptide segments is provided, including: (a) preparing a multi-phase solvent mixture, containing at least one organic solvent; (b) adding a first polypeptide containing a first amino acid having a first unprotected reactive group; (c) adding a second polypeptide containing a second amino acid having a second unprotected reactive group; and, (d) collecting the product polypeptide formed by a reaction between the first polypeptide with the ligation label; wherein the first unprotected reactive group and the second unprotected reactive group react to form a covalent bond between the first unprotected reactive group and the second unprotected reactive group.

In another embodiment of the present invention, a chemical intermediate composition is provided, containing: (a) a multiphase solvent mixture, containing at least two component solvents, wherein one of the at least two component solvents exhibits a dielectric strength of 4 to 12 while another of the at least two component solvents exhibits a higher dielectric strength and wherein at least one of the at least two component solvents is an organic solvent; (b) a first polypeptide containing a first amino acid having a first unprotected reactive group; and, (c) a ligation label containing a second amino acid having a second unprotected group.

In another embodiment of the present invention, a composition is provided, comprising: (a) a multiphase solvent mixture, wherein the solvent mixture containing at least one organic solvent; (b) a first polypeptide containing a first amino acid having a first unprotected reactive group; (c) a ligation label containing a second amino acid having a second unprotected group; and, (d) a product polypeptide formed by a reaction between the first polypeptide with the ligation label; wherein the first unprotected reactive group and the second unprotected reactive group react to form a covalent bond between the first unprotected reactive group and the second reactive group.

### BRIEF DESCRIPTION OF THE DRAWING

There are shown in the drawings certain exemplary embodiments of the present invention as presently preferred. It should be understood that the present invention is not limited to the embodiments disclosed as examples, and is capable of variation within the spirit and scope of the appended claims.

In the drawings,

FIG. 1 is a depiction of a cell membrane representing the dielectric properties thereof;

FIG. 2 is a depiction of a cell membrane model showing the penetration of a molecule through the membrane based on its position on the lipid chain;

FIG. 3 is a representation of a G-protein coupled receptor aligned within the cell membrane environment;

FIG. 4 is a graphical representation of the results of the experiments discussed in Example 1, herein;

FIG. 5 is a depiction of the transmembrane domain involved in Examples 2 and 3, herein;

FIG. 6 is a graphical representation of the results of the experiments discussed in Example 2, herein; and,

FIGS. 7-10 are graphical representations of the results of the experiments discussed in Example 3, herein.

### DEFINITIONS

The term “Amino Acids” as used herein and in the appended claims, includes the 20 genetically coded amino acids, rare or unusual amino acids that are found in nature and any of the non-naturally occurring and modified amino acids. Sometimes the term “Amino Acids” includes amino acid residues when in the context of a peptide, polypeptide or protein.

The term “Chemoselective chemical ligation” as used herein and in the appended claims is a chemically selective reaction involving covalent ligation of a first unprotected amino acid, peptide or polypeptide with a second amino acid, peptide or polypeptide and includes any chemoselective reaction chemistry that can be applied to ligation of unprotected peptide segments.

The term “Chromophore” as used herein and in the appended claims refers to chemical moiety which exhibits light absorption within the ultraviolet (250-400 nm) to the visible (400-700 nm) light regions of the spectrum, such as fluorophores, dyes and donor and acceptor moieties of a resonance energy transfer system. These chemical moieties may be naturally occurring or may be added to a biological molecule such as a peptide, polypeptide, carbohydrate or lipid.

The term “Cleavage Site” as used herein and in the appended claims refers to an amino acid sequence capable of being cleaved by a reagent comprising a chemical or protease that facilitates hydrolysis of a peptide bond between two amino acids of a target polypeptide.

The term “Hydrazone chemical ligation” as used herein and in the appended claims includes chemoselective reactions involving ligation of a first unprotected amino acid, peptide or polypeptide having a hydrazine moiety and a second unprotected amino acid, peptide or polypeptide having an aldehyde or ketone moiety resulting in the formation of a ligation product containing a hydrazone moiety at the ligation site. The backbone structure of a peptide or polypeptide product resulting form hydrazone forming chemical ligation is different from the backbone structure of a peptide or polypeptide occurring in nature or via recombinant expression.

The term “Ligation Label” as used herein and in the appended claims refers to a chemical moiety comprising one or more amino acids and can be a peptide or polypeptide.

The terms “Membrane polypeptide” (also “membrane peptide” and “membrane protein”) as used herein and in the appended claims includes polypeptides containing a hydrophobic moiety. Membrane polypeptides may be single or multi-pass transmembrane polypeptides or useful fragments thereof. Membrane polypeptides may include one or more extramembranous amino acid residues that preferentially interact with an aqueous phase, such as residues comprising an extracellular or intracellular loop.

The term “Native chemical ligation” as used herein and in the appended claims refers to chemoselective reactions involving ligation of a first unprotected amino acid, peptide or polypeptide and a second unprotected amino acid, peptide or polypeptide resulting in the formation of an amide bond having a backbone structure indistinguishable from that of a peptide or polypeptide occurring in nature or via recombinant expression.

The term “Oxazolidine chemical ligation” as used herein and in the appended claims refers to chemoselective reaction involving ligation of a first unprotected amino acid, peptide or polypeptide having an aldehyde or ketone moiety and a second unprotected amino acid, peptide or polypeptide having a 1-amino, 2-ol moiety resulting in the formation of an oxazolidine moiety at the ligation site. The backbone structure of a peptide or polypeptide product resulting from oxazolidine forming chemical ligation is distinguishable from that of a peptide or polypeptide occurring in nature or via recombinant expression.

The term “Oxime chemical ligation” as used herein and in the appended claims refers to chemoselective reaction involving ligation of a first unprotected amino acid, peptide or polypeptide having an amino-oxy moiety and a second unprotected amino acid, peptide or polypeptide having an aldehyde or ketone moiety resulting in the formation of an oxime moiety at the ligation site. The backbone structure of a peptide or polypeptide product resulting from oxime chemical ligation is distinguishable from that of a peptide or polypeptide occurring in nature or via recombinant expression.

The term “Peptide” as used herein and in the appended claims is a polymer of at least two monomers, wherein the monomers are amino acids, sometimes referred to as amino acid residues, which are joined together via an amide bond. Peptides may exhibit either a completely native amide backbone or an unnatural backbone or a mixture thereof. Peptides may be prepared by known synthetic methods, including solution synthesis, stepwise solid phase synthesis, segment condensation and convergent condensation. Peptides may be synthesized ribosomally in cell or in a cell free system, or generated by proteolysis of larger polypeptide segments. Peptides may also be synthesized by a combination of chemical and ribosomal methods.

The term “Polypeptide” as used herein and in the appended claims is a polymer comprising three or more monomers, wherein the monomers are amino acids, sometimes referred to as amino acid residues, which are joined together via an amide bond. Polypeptides may also be referred to as a protein. Polypeptides may include native amide bonds or any of the known unnatural peptide backbones or a mixture thereof. Polypeptides typically range in size from 3 to 1000 amino acid residues, from 3-100 amino acid residues, from 10-60 amino acid residues and from 20-50 amino acid residues. Segments or all of the polypeptide can be prepared by known synthetic methods, including solution synthesis, stepwise solid phase synthesis, segment condensation, and convergent condensation. Segments or all of the polypeptide can also be prepared ribosomally in a cell or in a cell-free translation system, or generated by proteolysis of larger polypeptide segments. Polypeptides may also be synthesized by a combination of chemical and ribosomal methods.

The term “Target polypeptide” as used herein and in the appended claims is a polypeptide with which a ligation label is reacted in the methods of the present invention.

The term “Thiazolidine chemical ligation” as used herein and in the appended claims is a chemoselective reaction involving ligation of a first unprotected amino acid, peptide or polypeptide having an aldehyde or ketone moiety and a second unprotected amino acid, peptide or polypeptide having a 1-amino, 2-thiol moiety resulting in the formation of a thiazolidine moiety at the ligation site. The backbone structure of a peptide or polypeptide product resulting from thiazolidine chemical ligation is distinguishable from that of a peptide or polypeptide occurring in nature or via recombinant expression.

The term “Thioester chemical ligation” as used herein and in the appended claims is a chemoselective reaction involving ligation of a first unprotected amino acid, peptide or polypeptide and a second unprotected amino acid, peptide or polypeptide resulting in the formation of a thioester bond at the ligation site. The backbone structure of a peptide or polypeptide product resulting from thioester chemical ligation is distinguishable from that of a peptide or polypeptide occurring in nature or via recombinant expression.

The term “Thioether chemical ligation” as used herein and in the appended claims is a chemoselective reaction involving ligation of a first unprotected amino acid, peptide or polypeptide and a second unprotected amino acid, peptide or polypeptide resulting in the formation of a thioether bond at the ligation site. The backbone structure of a peptide or polypeptide product resulting from thioether chemical ligation is distinguishable from that of a peptide or polypeptide occurring in nature or via recombinant expression.

### DETAILED DESCRIPTION

Embodiments of the present invention will now be described with reference to the drawings. This detailed description is of a presently contemplated mode of carrying out the invention is not intended in a limiting sense, and it is made solely for the purpose of illustrating the general principles of the invention.

The present invention relates to methods for chemical ligation and synthesis of polypeptides and proteins, including, but by no means limited to, membrane polypeptides or membrane polypeptide domains, in a multiphase solvent mixture comprising at least two component solvents, wherein the components of the multiphase solvent mixture have different dielectric strengths. The present invention also relates to compositions produced using the methods of the present invention and assays performed using those compositions.

Chemical ligation and synthesis methods of the present invention involve chemoselective ligation of a ligation label to a target polypeptide in a multiphase solvent mixture comprising at least two component solvents, wherein the components of the multiphase solvent mixture have different dielectric strengths. The target polypeptide and ligation label components possess unprotected reactive groups that selectively react to yield a covalent bond at the ligation site, also referred to as a chemoselective ligation site. The multiphase solvent mixture provides an environment in which the hydrophobic regions of the ligation label and the target polypeptide reside in one phase of the solvent mixture while the reactive hydrophilic regions reside in a different phase. This partitioning of the reactive sites into a different phase of the multiphase solvent mixture maximizes exposure and collision of the reactive sites enabling high yield coupling thereof. In this way, the multiphase solvent mixtures of the present invention provide a cell membrane mimetic.

Product proteins or polypeptide domains produced using the methods of the present invention may include (a) totally synthetic products wherein all the ligation components are man-made through chemical synthesis (i.e., ribosomal-free synthesis); and (b) semi-synthetic products wherein part of the ligation component is made by biological synthesis (i.e., ribosomally in a cell or cell-free translation system) and part is made by chemical synthesis.

The methods of the present invention lend themselves to a variety of chemistries. Specifically, any chemoselective reaction chemistry applicable for the ligation of unprotected peptide segments is suitable for use with the methods of the present invention. Suitable chemistries include, but are by no means limited to, native chemical ligation,²² extended general chemical ligation, oxime-forming chemical ligation,²⁴ thioester forming ligation,²⁵ thioether forming ligation,²⁶ hydrazone forming ligation,²⁷ thiazolidine forming ligation and oxazolidine forming ligation.²⁸ ²² See, eg., Dawson, et al., Science (1994) 266:776-779 and WO 96/34878. ²³ See, e.g., WO 98/28434. ²⁴ See, e.g., Rose, et al., J. Amer. Chem. Soc. (1994) 116:30-33. ²⁵ See, e.g., Schnolzer, et al., Science (1992) 256:221-225. ²⁶ See, e.g., Englebretsen, et al., Tet. Letts. (1995) 36(48):8871-8874. ²⁷ See, e.g., Gaertner, et al., Bioconj. Chem. (1994) 5(4):333-338. ²⁸ See, e.g., Zhang, et al., Proc. Natl. Acad Sci. (1998) 95(16):9184-9189 and WO 95/00846.

For native chemical ligation, the target polypeptide and the ligation label should provide a component pairing which is capable of chemically reacting to yield a native peptide bond at the ligation site. An example of such a component pairing would be one in which one of the components exhibits a cysteine having an unprotected amino group while the other component exhibits an amino acid having an unprotected alpha-thioester group.

For oxime-forming chemical ligation, the target polypeptide and ligation label should provide a component pairing in which one of the components exhibits an unprotected amino acid having an aldehyde or ketone moiety while the other component exhibits an unprotected amino acid having an amino-oxy moiety. Such groups are capable of chemically reacting to yield a ligation product with an oxime moiety at the ligation site.

For thioester-forming chemical ligation, the target polypeptide and ligation label should provide a component pairing in which one of the components exhibits an unprotected amino acid having a haloacetyl moiety while the other component exhibits an unprotected amino acid having an alpha-thiocarboxylate moiety. Such groups are capable of chemically reacting to yield a ligation product with a thioester moiety at the ligation site.

For thioether-forming chemical ligation, the target polypeptide and ligation label should provide a component pairing in which one of the components exhibits an unprotected amino acid having a haloacetyl moiety while the other component exhibits an unprotected amino acid having an alkyl thiol moiety. Such groups are capable of chemically reacting to yield a ligation product with a thioether moiety at the ligation site.

For hydrazone-forming chemical ligation, the target polypeptide and ligation label should provide a component pairing in which one of the components exhibits an unprotected amino acid having an aldehyde or ketone moiety while the other component exhibits an unprotected amino acid having a hydrazine moiety. Such groups are capable of chemically reacting to yield a ligation product with a hydrazone moiety at the ligation site.

For thiazolidine-forming chemical ligation, the target polypeptide and ligation label should provide a component pairing in which one of the components exhibits an unprotected amino acid having a 1-amino, 2-thiol moiety while the other component provides an unprotected amino acid having an aldehyde or a ketone moiety. Such groups are capable of chemically reacting to yield a ligation product with a thiazolidine moiety at the ligation site.

For oxazolidine-forming chemical ligation, the target polypeptide and ligation label should provide a component pairing in which one of the components exhibits an unprotected amino acid having a 1-amino, 2-hydroxyl moiety while the other component provides an unprotected amino acid presenting an aldehyde or a ketone moiety. Such groups are capable of chemically reacting to yield a ligation product with an oxazolidine moiety at the ligation site.

Any combination of ligation components adapted for chemoselective chemical ligation to yield a target polypeptide product having a covalent bond at the ligation site are considered part of the invention, provided that the reaction of the reaction ligation components takes place in a multiphase solvent mixture of the present invention, which mixture contains at least two component solvents, wherein the component solvents of the multiphase solvent mixture have different dielectric strengths.

Multiphase solvent mixtures suitable for use with the present invention include mixtures of at least two component solvents, wherein one of the at least two component solvents exhibits a dielectric strength of 4 to 12 while another of the at least two component solvents exhibits a higher dielectric strength. In some embodiments of the present invention, the at least two component solvents will exhibit dielectric strengths that differ by less than about 70, less than about 30.

Multiphase solvent mixtures suitable for use with the present invention include mixtures of at least two component solvents which form at least two phases when combined. For the purposes of the present invention, a mixture of solvents is considered to form at least two phases if either (a) an interfacial meniscus forms or (b) an emulsion forms subsequent to the intermixing of the solvent components. It will be recognized by one skilled in the art that the solvents forming two or more phases may exhibit some degree of partial miscibility. Where a given coupling of component solvents results in a pronounced degree of partial miscibility, the proportions of the component solvents in the separate phases may be taken into account in designing a given multiphase solvent mixture cell membrane mimetic.

Multiphase solvent mixtures suitable for use with the present invention include, but are by no means limited to, mixtures containing (a) a first component solvent selected from the group consisting of: amyl alcohol, heptanol, octanol, nonanol, decanol, hexanol; and (b) a second component solvent selected from the group consisting of: dimethyl formamide (DMF), water, methanol, ethanol and isopropanol, dioxane, tetrahydrofuran.

Multiphase solvent mixtures suitable for use with the present invention may include emulsions. Such multiphase solvent mixtures may further contain compatible emulsifiers. Emulsifiers suitable for use with the present invention include phospholipids (such as lecithin), block copolymers (such as a poloxamer copolymer), and mixtures thereof.

Ligation label components suitable for use with the present invention include components containing one or more amino acids, peptides and polypeptides. Suitable ligation labels should contain at least one amino acid with a reactive group capable of reacting with and forming a covalent bond with a reactive group exhibited by the target membrane polypeptide. In some embodiments of the present invention, the ligation label may contain a membrane polypeptide component. Such embodiments are useful for the modular ligation and synthesis of multipass transmembrane polypeptides. The ligation label may contain native and/or unnatural peptide backbone structure or unnatural amino acid residues or other chemical differences from native peptide sequences. For example, the ligation label may contain an unnatural amino acid including a chromophore or other detectable moiety compatible with chemical ligation of the ligation label and the target membrane polypeptide.

In some embodiments of the present invention, one or more chemical tags may be incorporated into the membrane polypeptide and/or the ligation label. Chemical tags may be used to facilitate the synthesis process, purification, anchoring to a support matrix, detection and the like.

Target polypeptides suitable for use with the present invention include polypeptides containing a hydrophobic moiety. The target polypeptides may be membrane polypeptides. The target polypeptide may be a fragment of: Interferon alfa-n1, interferon beta-1a, interferon beta-2b, G-CSF, Darbepoetin, erythropoetin, anakinra, oprelvekin (IL-11), Rebif, Filgrastim, beta-endorphin, prolactin, growth hormone, fibroblast growth factor and epidermal growth factor.

Target polypeptide and ligation label component pairings suitable for use with the present invention can be derived from virtually any known membrane protein system including viral, eukaryotic, prokaryotic and archaebacterial systems, including psychrophilic, mesophilic and thermophilic organisms. In some embodiments, the membrane polypeptide and ligation label component pairings are derived from the two major classes of membrane proteins—those that insert alpha-helices into the lipid bilayer and those that form pores with beta-barrel strands. Examples of suitable membrane proteins include membrane associated receptors; transporter proteins; enzymes; and immunogens. Membrane proteins of particular interest include enzyme-linked receptors, fibronectin-like receptors, the seven transmembrane receptors (i.e., G-protein coupled receptors) and the ion channel receptors. The membrane proteins of most particular interest are the membrane protein superfamily of G-protein coupled receptors. G-protein coupled receptors have been characterized as including seven conserved hydrophobic stretches of about 20 to 30 amino acids which connect at least eight divergent hydrophilic loops. The family of G-protein coupled receptors include, but are by no means limited to, dopamine receptors, calcitonin, adrenergic, endothelin, cAMP, adenosine, muscarinic, acetylcholine, serotonin, histamine, thrombin, kinin, follicle stimulating hormone, opsins, endothelial differentiation gene-1, rhodopsins, odorant and cytomegalovirus receptors.

The reaction components used in the methods of the present invention—the target polypeptide and the ligation label—may be synthesized using any number of conventional methods. Such conventional methods include chemical methods, biological methods and combinations thereof. For example, a nucleic acid sequence coding for part or all of a ligation component may be expressed in a host cell, and recovered using standard techniques.

Polypeptides for use in the target polypeptides and ligation labels of the present invention may in some cases be produced using standard biological techniques including, but not limited to: (a) cells that naturally express a given polypeptide and (b) transfection and transformation of a host cell with DNA encoding a polypeptide of interest. For instance, a polymerase chain reaction (PCR) based strategy may be used to clone a target DNA sequence encoding all or part of a given membrane polypeptide.²⁹ PCR can also be used for cloning through differential and subtractive approaches to cDNA analysis, performing and optimizing long-distance PCR, cloning unknown neighboring DNA and using PCR to create and screen libraries. PCR may also be used to create site-specific and random mutations in DNA encoding a given target membrane polypeptide or ligation label. ²⁹ See, eg., “PCR Cloning Protocols: From Molecular Cloning to Genetic Engineering,” B. A. White, ed., Humana Press, Methods in Molecular Biology, Vol. 67, 1997.

Once a clone for producing a given polypeptide is identified, it may be expanded and used to produce large quantities of the polypeptide. The polypeptide so prepared my then be purified using conventional techniques well-known in the art including, but by no means limited to, immunoaffinity purification, chromatographic methods including high performance liquid chromatography or cation exchange chromatography, affinity chromatography based on affinity of the polypeptide for a particular ligand, immunoaffinity purification using antibodies and the like.³⁰ ³⁰ See, eg., Ohlendieck, K., Methods Mol Biol. (1996) 59:293-304, 313-322 and Josic D, et al., Methods Enzymol. (1996), 271:113-134.

In some embodiments of the present invention, target polypeptides and ligation labels may be produced with the help of well known conventional chemical synthesis techniques. Such techniques may be used to produce peptides and polypeptides having a native peptide backbone structure.³¹ Such techniques may also be used to produce peptides and polypeptides having an unnatural peptide backbone.³² Such techniques may also include extended general chemical ligation and synthesis as disclosed in WO 98/28434. ³¹ See, eg., WO 96/34878; Dawson, et al., Science (1994) 266:77-779 and Tam, et al., Proc. Natl. Acad. Sci. USA (1995) 92:12485-12489. ³² See, eg., Schnolzer, et al., Science (1992) 256:221-225; Rose, et al., J. Am. Chem. Soc. (1994) 116:30-34; Liu, et al., Proc. Nati. Acad. Sci. USA (1994) 91:6584-6588; Englebretsen, et al., Tet. Letts. (1995) 36(48):8871-8874; Gaertner, et al., Bioconj. Chem. (1994) 5(4):333-338; Zhang, et al., Pro. Nati. Acad. Sci. USA (1998) 95(16):9184-9189 and WO 95/00846.

In some instances, the methods used to produce the polypeptides found in the target polypeptides and ligation labels will be dictated by the inherent properties of the polypeptides involved. For example, some polypeptides including hydrophobic domains are difficult to impossible to produce with conventional techniques. Accordingly, some combination of conventional techniques and the techniques of the present invention may be necessary to produce some of the polypeptides.

Ligation components suitable for use with the present invention may contain moieties which facilitate purification and/or detection of the final product. For example, the ligation components may include purification handles or tags that bind to an affinity matrix. Many of these moieties are known and techniques for incorporating them into ligation components are also known, for example, using post-synthesis chemical modification and/or during synthesis.³³ ³³ See, eg., Protein Purification Protocols, (1996), Doonan, S., ed., Humana Press Inc.; Schriemer, et al., Anal. Chem. (1998) 70(8):1569-1575; Evangelista, et al., J. Chromatogr. B. Biomed. Sci. AppI. (1997) 699(1-2):383-401; Kaufmann, M., J. Chromatogr. B. Biomed. Sci. AppI. (1997) 699(1-2):347-369; Nilsson, et al, Protein Expr. Purif (1997)11(1):1-16; Lanfermeijer, et al., Protein Expr. Purif. (1998) 12(1): 29-37.

In some embodiments it may be desirable to include in a ligation component one or more unnatural amino acids having a chemical moiety that imparts a particular property which symplifies purification. These purification sequences may be incorporated into the ligation component by recombinant DNA techniques. In some embodiments it may be desirable to include a chemical or protease cleavage site in the ligation component to remove a tag, depending on the tag and the intended end use. In some embodiments an unnatural amino acid or chemically modified amino acids such as a chromophore, hapten or biotinylated moiety detectable by fluorescence spectroscopy, immunoassays and/or MALDI mass spectrometry may be included in the ligation component to ease detection.

One skilled in the art will know how to confirm the homogeneity and the structural identity of the desired target polypeptide and ligation label components by a variety of means including immunoassays, fluorescence spectroscopy, gel electrophoresis, HPLC using either reverse phase or ion exchange columns, amino acid analysis, mass spectrometry, crystallography, NMR and the like. The position of amino acid modifications, insertions and/or deletions can be identified by sequencing with either chemical methods (Edman chemistry) or tandem mass spectrometry.

The sequence of addition of the ligation label and target polypeptide to the multiphase solvent mixture is not critical. That is, in some embodiments, the ligation label may be added to the multiphase solvent mixture first, followed by the addition of the target polypeptide. In some other embodiments, the target polypeptide may be added to the multiphase solvent mixture first, followed by the addition of the ligation label. In still some other embodiments, the target polypeptide and the ligation label may be simultaneously added to the multiphase solvent mixture.

When the target polypeptide and ligation label are reconstituted together, one or more of the ligation components can include a linker or capping sequence having one or more cleavage sites positioned for generating a chemically reactive ligation site moiety upon cleavage, and thus used to control the presence or absence of reactive groups needed for the ligation reaction. Additionally, reagents or buffer conditions can be utilized to control the presence and reactivity of such reactive groups.

Reaction conditions for the methods of the present invention are selected to maintain the desired interaction of the target polypeptide and the ligation label in the multiphase solvent mixture. Process conditions playing a role in the subject reactions may include temperature, pH, make up of the multiphase solvent mixture, ion concentration, reducing or oxidizing reagents, ratios of reactants, etc. One skilled in the are will recognize how to identify optimal process conditions by making routine adjustments thereto. One of ordinary skill will recognize that these conditions are selected based on a given ligation chemistry utilized, and thus conditions appropriate to that chemistry. Such conditions and ranges of conditions are readily determined by one of ordinary skill in the art.

One skilled in the art will know how to use any one of a variety of techniques to monitor the formation of the desired ligation product. Such techniques include assays that detect activity of the desired ligation product, fluorescence spectroscopy, mass spectrometry, affinity matrix and ligand binding assays, NMR, circular dichroism (CD), scanning densitometry, calorimetry, and various chromatography techniques such as electrophoresis, affinity chromatography, HPLC using either reverse phase or ion exchange columns and the like.

### EXAMPLE 1

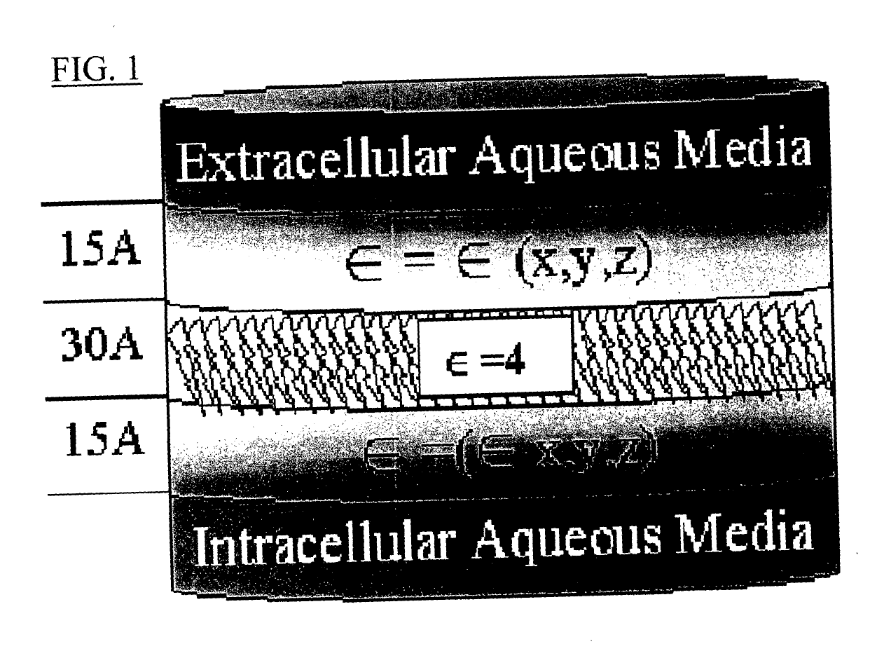

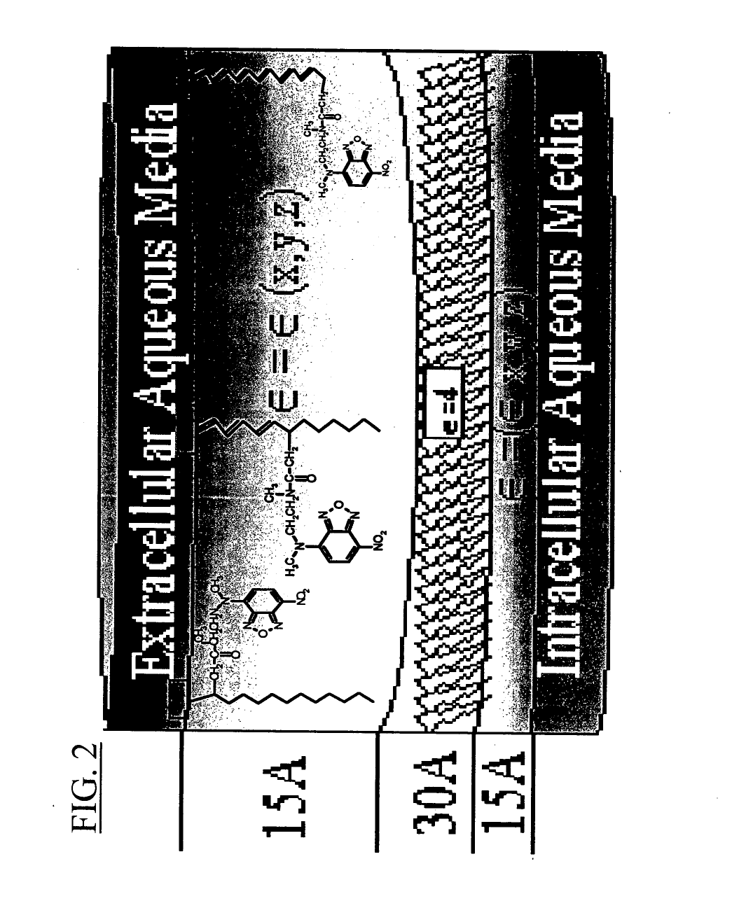

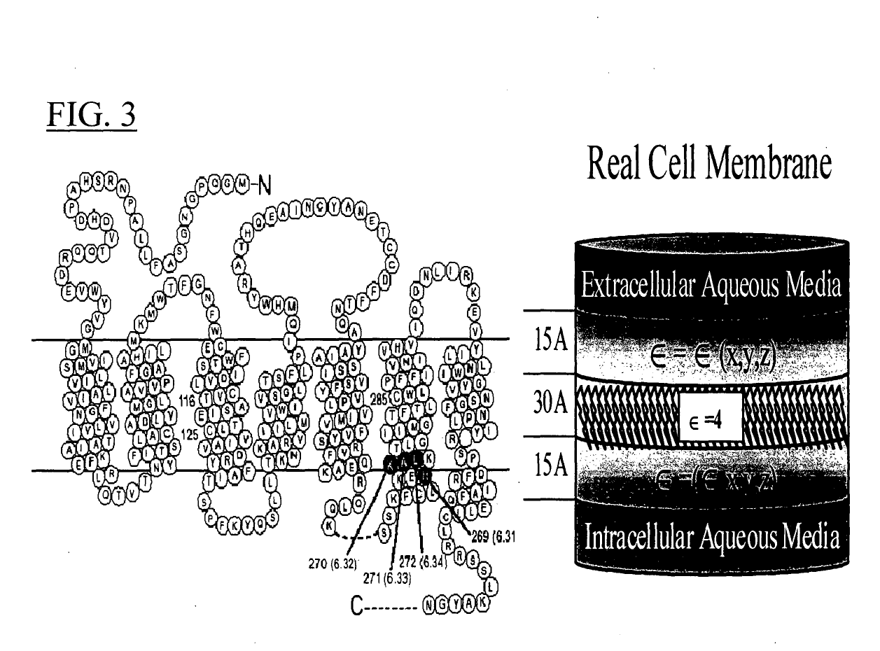

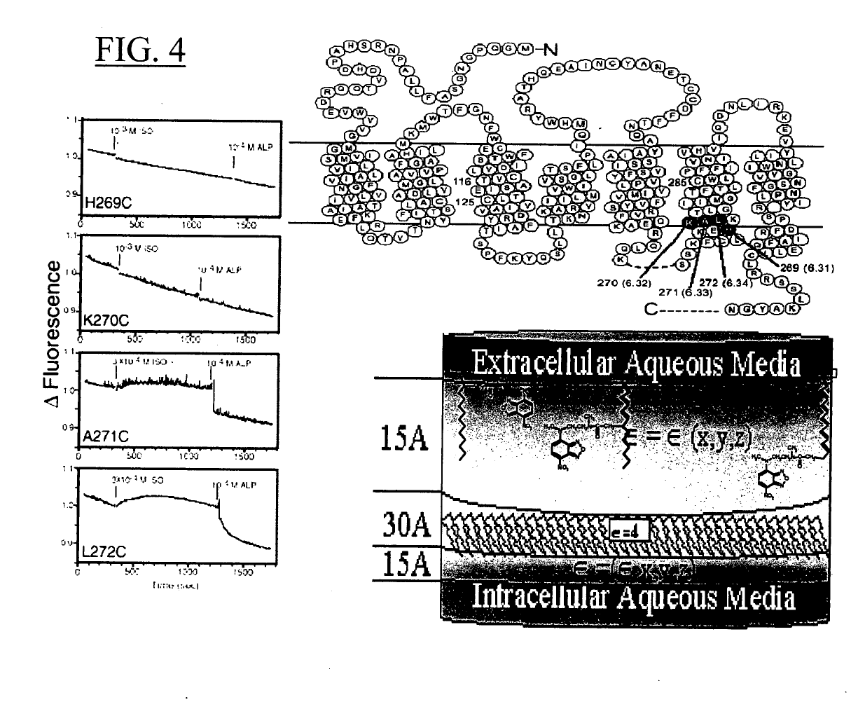

A mathematical model of the cell membrane as depicted in FIG. 1 was created. A model of helix 6 of the receptor was constructed. The dynamics of this portion of the receptor was computationally modeled. Computationally, the dynamics of the active state of the receptor (receptor with drug molecule bound) resulted in the four blackened residues of helix 6 collectively moving up into a relatively more hydrophobic region (lower dielectric) of the cell membrane. To test the computational predictions four individual experiments were carried out. Residue 269 was mutated to a cysteine and then a flourophore was chemically attached to this amino acid. The fluorophore was continually excited (the constant negative slope of the line is due to bleaching of the fluorophore) and exposed to an agonist (ISO), which converted the receptor to its active form followed by exposure to an antagonist (ALP), which converted the receptor back to its inactive form. The same experiments were performed on residues 270, 271 and 272. The results of these experiments are depicted in FIG. 4. Fluorescence lifetime increases when the fluorophore moves into a more hydrophobic environment. The experimental finding that residues 271 and 272 dramatically increase their fluorescence lifetime upon agonist binding and decrease lifetime upon antagonist binding is consistent with the predictions of the simulation and was a priori experimentally unexpected. These computational predictions and experimental validation were published in J. Biol. Chem Mar. 23, 2001; 276(12): 9279-90, Jensen A D; Guarnieri F; Rasmussen S G; Asmar F; Ballesteros J A; Gether U. These results validate the membrane model.

### EXAMPLE 2

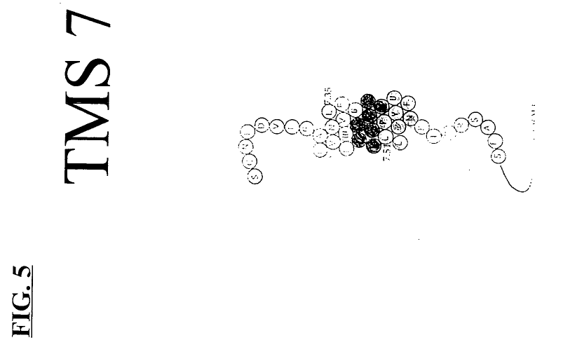

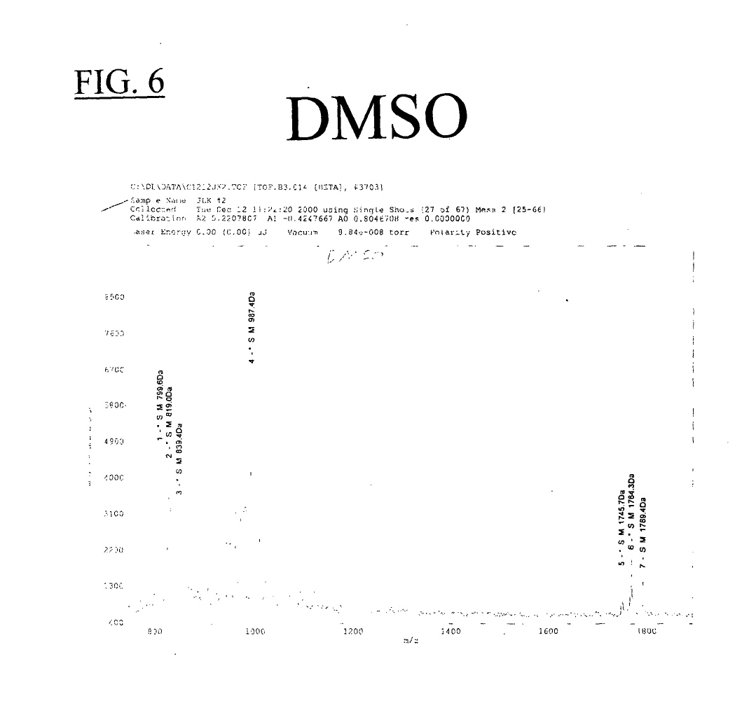

G-protein coupled receptors span the cell membrane seven times. As a demonstration of the principle, the seventh transmembrane domain of a serotonin receptor was synthesized. FIG. 5 depicts the residues of this transmembrane domain. A commercial peptide house synthesized the red portion and the blue portion. None of the commercial sources contacted was able to synthesis the whole peptide (contiguous string of the red and blue) and all of commercial sources contacted had great difficulty synthesizing the individual read and blue peptides. The red and blue regions were chosen because they are embedded within the most hydrophobic region of the membrane and are thus known to be the most difficult to synthesize. The first experiment was an attempt to couple the red and blue peptide in DMSO. The red peptide weighs approximately 700 Daltons and the blue peptide weighs about 1000 Daltons. The combined weight of the red and blue peptide covalently linked together is about 1740 Daltons. An electrospray mass spectrum of the results of this reaction shows virtually no product and all starting material. See FIG. 6.

### EXAMPLE 3

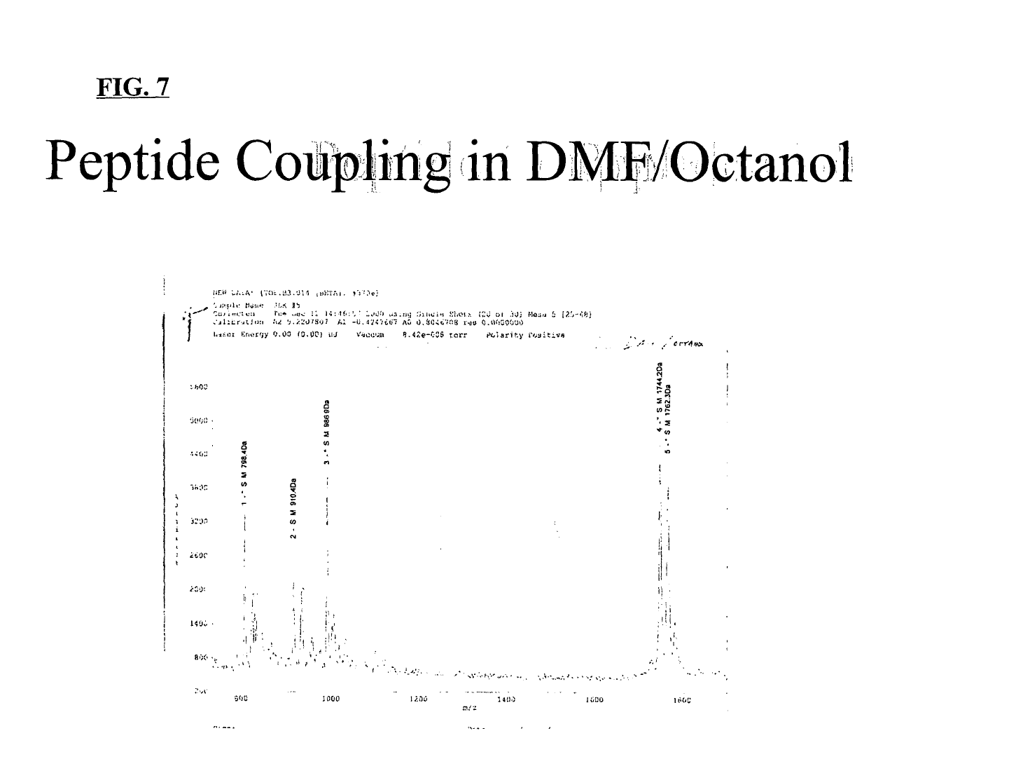

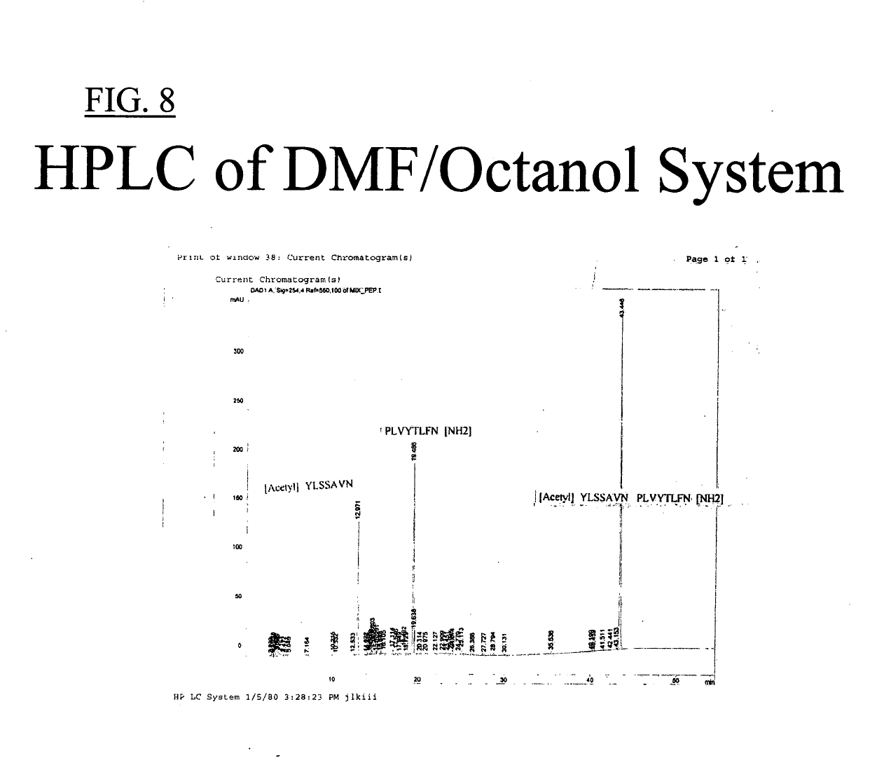

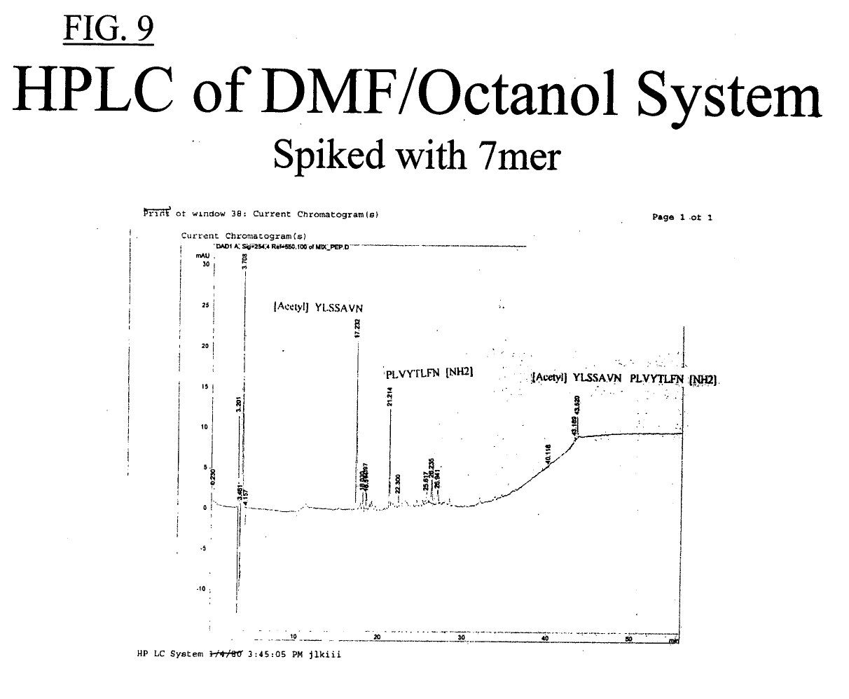

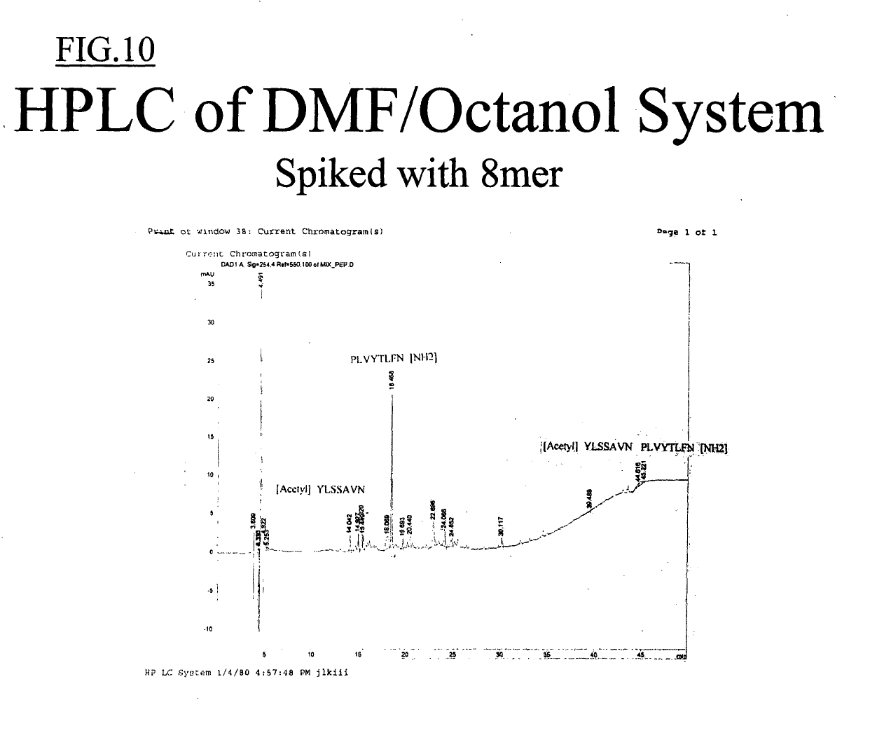

The same coupling reaction discussed in Example 3 was performed in a multiphase solvent mixture of the present invention (DMF/Octanol). Using conventional native chemical ligation techniques, the red and blue polypeptides were reacted in the DMF/octanol multiphase solvent mixture. As the reaction progressed, mixture was initially cloudy but was observed to become clear as the reaction progressed. FIG. 7 is an electrospray mass spectrum showing that significant product was created. A careful examination of the spectra show a sharp doublet around 1740 separated by 22 mass units. The higher molecular weight peak is a sodium ion replacing a proton. To quantify the yield HPLC was performed. The relative heights of the reactant and product peaks shown in FIG. 8 indicate that the reaction has gone to significant completion. As a check on the results HPLC spiking experiments were performed. FIG. 9 shows the results of redoing the HPLC of the reaction with added red peptide and FIG. 10 shows the results of the same experiment with added blue peptide. In both cases it is seen that the reactant peaks identified in the initial HPLC are significantly elevated when either reactant is added confirming peak assignment.

The present invention may also be used to provide a solvent system for use with a conventional solid-phase peptide synthesis. The present invention would be of particular usefulness in such systems when a hydrophobic segment of a peptide is being made, or when a lengthy segment including a highly hydrophobic region has already been formed on a support. For such syntheses, agitation could be used to expose the support-bound nascent peptides to two or more of the solvent phases.

The present invention having been disclosed in connection with the foregoing embodiments, such additional embodiments will now be apparent to persons skilled in the art. The present invention is not intended to be limited to the embodiments specifically mentioned, and accordingly reference should be made to the appended claims rather than the foregoing discussion, to assess the spirit and scope of the present invention in which exclusive rights are claimed.

All publications and patent applications mentioned in this specification are herein incorporated by reference to the same extent as if each individual publication or patent application was specifically and individually indicated to be incorporated by reference. The invention now being fully described, it will be apparent to one of ordinary skill in the art that many changes and modifications can be made thereto without departing from the spirit or scope of the appended claims.

We claim:

1. A method for synthesis of a product polypeptide, comprising:
(a) preparing a multiphase solvent mixture, wherein the solvent mixture comprises at least one organic solvent;
(b) adding a first polypeptide comprising a first amino acid having a first unprotected reactive group;
(c) adding a ligation label comprising a second amino acid having a second unprotected reactive group; and
(d) collecting the product polypeptide formed by a reaction between the first polypeptide with the ligation label;
wherein the first unprotected reactive group and the second unprotected reactive group react to form a covalent bond between the first unprotected reactive group and the second unprotected reactive group.

2. The method of claim 1, wherein the multiphase solvent mixture is a two phase mixture.

3. The method of claim 2, wherein the multiphase solvent mixture comprises at least two component solvents, wherein one of the at least two component solvents exhibits a dielectric strength of 4 to 12 while another of the at least two component solvents exhibits a higher dielectric strength.

4. The method of claim 1, wherein the multiphase solvent mixture is an emulsion.

5. The method of claim 4, wherein the multiphase solvent mixture further comprises an emulsifier.

6. The method of claim 5, wherein the emulsifier is selected from the group consisting of phospholipids, block copolymers, and mixtures thereof.

7. The method of claim 1, wherein the first polypeptide is selected to have a sequence of membrane-incorporated polypeptide segment.

8. The method of claim 7, wherein the ligation label is a second polypeptide and wherein the second polypeptide is selected to have a sequence of membrane-incorporated polypeptide segment.

9. The method of claim 8, wherein the first polypeptide is selected from the group consisting of a membrane polypeptide, a transmembrane polypeptide, a receptor and useful fragments thereof.

10. The method of claim 9, wherein the first polypeptide and the second polypeptide are portions of a G-protein coupled receptor, and the linked polypeptides form a larger portion of the G-protein coupled receptor.

11. The method of claim 1, wherein the multiphase solvent mixture comprises at least two organic solvents.

12. The method of claim 11, wherein the multiphase solvent mixture comprises DMF and octanol.

13. A method for chemoselective chemical ligation of a product polypeptide, comprising:
(a) preparing a multiphase solvent mixture, comprising at least one organic solvent;
(b) adding a first polypeptide comprising a first amino acid having a first unprotected reactive group;
(c) adding a ligation label comprising a second amino acid having a second unprotected reactive group; and,
(d) collecting the product polypeptide formed by a reaction between the first polypeptide with the ligation label;
wherein the first unprotected reactive group undergoes chemoselective chemical ligation with the second unprotected reactive group to form a covalent bond between the first unprotected reactive group and the second unprotected reactive group.

14. The method of claim 13, wherein the chemoselective ligation is selected from the group of native chemical ligation, oxime-forming ligation, thioester-forming ligation, thioether-forming ligation, hydrazone-forming ligation, thiazolidine-forming ligation and oxazolidine-forming ligation.

15. The method of claim 14, wherein the first polypeptide is selected to have a sequence of membrane-incorporated polypeptide segment.

16. The method of claim 13, wherein the multiphase mixture is a two phase mixture.

17. The method of claim 16, wherein the multiphase solvent mixture comprises at least two component solvents, wherein one of the at least two component solvents exhibits a dielectric strength of 4 to 12 while another of the at least two component solvents exhibits a higher dielectric strength.

18. The method of claim 13, wherein the multiphase solvent mixture is an emulsion.

19. The method of claim 18, wherein the multiphase solvent mixture further comprises an emulsifier.

20. The method of claim 19, wherein the emulsifier is selected from the group consisting of phospholipids, block copolymers, and mixtures thereof.

21. The method of claim 13, wherein the first polypeptide is selected to have a sequence of membrane-incorporated polypeptide segment.

22. The method of claim 21, wherein the ligation label is a second polypeptide and wherein the second polypeptide is selected to have a sequence of membrane-incorporated polypeptide segment.

23. The method of claim 22, wherein the first polypeptide is selected from the group consisting of a membrane polypeptide, a transmembrane polypeptide, a receptor and useful fragments thereof.

24. The method of claim 23, wherein the first polypeptide and the second polypeptide are portions of a G-protein coupled receptor, and the linked polypeptides form a larger portion of the G-protein coupled receptor.

25. The method of claim 13, wherein the multiphase solvent mixture comprises at least two organic solvents.

26. The method of claim 25, wherein the multiphase solvent mixture comprises DMF and octanol.

27. A method of assembling polypeptide segments, comprising:
(a) preparing a multi-phase solvent mixture, comprising at least one organic solvent;
(b) adding a first polypeptide comprising a first amino acid having a first unprotected reactive group;
(c) adding a second polypeptide comprising a second amino acid having a second unprotected reactive group; and,
(d) collecting the product polypeptide formed by a reaction between the first polypeptide with the ligation label;
wherein the first unprotected reactive group and the second unprotected reactive group react to form a covalent bond between the first unprotected reactive group and the second unprotected reactive group.

28. A chemical intermediate composition, comprising:
(a) a multiphase solvent mixture, comprising at least two component solvents, wherein one of the at least two component solvents exhibits a dielectric strength of 4 to 12 while another of the at least two component solvents exhibits a higher dielectric strength and wherein at least one of the at least two component solvents is an organic solvent;
(b) a first polypeptide comprising a first amino acid having a first unprotected reactive group; and,
(c) a ligation label comprising a second amino acid having a second unprotected group.

29. A composition, comprising:
(a) a multiphase solvent mixture, wherein the solvent mixture comprises at least one organic solvent;
(b) a first polypeptide comprising a first amino acid having a first unprotected reactive group;
(c) a ligation label comprising a second amino acid having a second unprotected group; and,
(d) a product polypeptide formed by a reaction between the first polypeptide with the ligation label;
wherein the first unprotected reactive group and the second unprotected reactive group react to form a covalent bond between the first unprotected reactive group and the second reactive group.
